(19) United States
(12) Patent Application Publication
(10) Pub. No.: US 2022/0184255 A1
(43) Pub. Date: Jun. 16, 2022

(54) Personal Sanitation Sprayer

(71) Applicant: Alexander Kim, Scarsdale, NY (US)

(72) Inventor: Alexander Kim, Scarsdale, NY (US)

(21) Appl. No.: 17/524,822

(22) Filed: Nov. 12, 2021

Related U.S. Application Data
(60) Provisional application No. 63/124,153, filed on Dec. 11, 2020.

(51) Int. Cl. A61L 2/18; B05B 11/00; G01F 23/02; G04B 47/00; A61L 2/10

(52) U.S. Cl. CPC A61L 2/18; B05B 11/0037; A61L 2202/15; G04B 47/00; A61L 2/10; G01F 23/02

(57) ABSTRACT

The present invention relates generally to the field of sanitizing devices. More specifically, the present invention relates to a wrist-worn sanitation sprayer having hollow body with a side surface with an opening sized and configured to fit around a wrist of a wearer. The top surface has a pump-action spray nozzle and an internal tubing. A disinfectant spray is stored within a reservoir within the body and reaches the nozzle via the internal tubing.

### CROSS-REFERENCE TO RELATED APPLICATION

The present application claims priority to, and the benefit of, U.S. Provisional Application No. 63/124,153, which was filed on Dec. 11, 2020, and is incorporated herein by reference in its entirety.

### FIELD OF THE INVENTION

The present invention relates generally to the field of sanitizing devices. More specifically, the present invention relates to a personal bracelet sanitation sprayer having a hollow body that has a side surface with a continuous opening, a top surface with a spray nozzle and an internal tubing. The continuous opening of the side surface allows the device to be worn around the wrist of the user. Further, a disinfectant spray is stored within the body and reaches the nozzle via the internal tubing. When desired, a user can press down on the nozzle to spray the disinfectant spray onto nearby surfaces. Therefore, the device allows a user to easily and conveniently sanitize and disinfectant everyday surfaces he or she comes in contact with, without having to carry additional sanitizing wipes or spray bottles. Accordingly, the present disclosure makes specific reference thereto. Nonetheless, it is to be appreciated that aspects of the present invention are also equally applicable to other like applications, devices and methods of manufacture.

### BACKGROUND

Many public surfaces are touched by numerous individuals on a daily basis, such as doorknobs, shopping cart handles, push bars, etc. that may be covered in billions of harmful pathogens, germs, viruses and bacteria. These areas are not frequently cleaned, and as a result an individual may grasp or touch a surface and contract and/or transfer any or all of the harmful pathogens, germs, viruses and bacteria on the surface to themselves and/or others. As a result, serious illness can then occur, including the spread of deadly viruses, illness and sickness. In order to combat the spread of harmful pathogens, germs, viruses and bacteria, a user typically will carry sanitizing spray or sanitizing wipes to sanitize various surfaces with which they come in contact.

However, both options are suboptimal, as the individual must store the wipes or spray containers within the pockets, purse or bag of the user. This is undesirable, as the spray or wipes take up space that could otherwise be used to store other necessary items, or may leak or spill into the area they are stored. Further, sanitizing wipes must be thrown away after use, thereby creating significant waste in landfills. However, in certain scenarios, trash cans may not be present, which then requires that the wipes be stored on an individual's person or in the purse or pocket until a trash can is found, or the user may just throw the used wipe on the ground, which is also undesirable and irresponsible.

Therefore, there exists a long-felt need in the art for a device that allows a user to easily sanitize every day and other surfaces with which they readily come into contact. There also exists a long-felt need in the art for a sanitation spray bracelet device that allows a user to easily sanitize everyday and other surfaces, and that can also be conveniently worn on the body of the user, such that the device does not have to be stored in a pocket, purse or bag. Further, there exists a long-felt need in the art for a bracelet sanitation sprayer that does not require a user to discard the device after use, in contrast to sanitizing wipes and spray bottles when they are finished.

The subject matter disclosed and claimed herein, in one embodiment thereof, comprises a bracelet sanitation sprayer. The device has a hollow body with a side surface with an opening, a top surface having a spray nozzle, and an internal tubing. The opening of the side surface allows the device to be worn around the wrist of the user. Further, a disinfectant spray is stored within a reservoir within the body, and is distributed by the nozzle via the internal tubing. When desired, a user can press down on the nozzle which draws material from the reservoir to spray the disinfectant spray onto nearby surfaces. Therefore, the invention allows a user to easily and conveniently sanitize and disinfect everyday surfaces with which they come into contact with.

In this manner, the bracelet sanitation sprayer of the present invention accomplishes all of the forgoing objectives, and allows a user to easily sanitize every day and other surfaces with which they come into contact. Further, the invention can be worn on the body of the user. As a result, the device does not have to be stored in a pocket, purse or bag. Further, the device does not require a user to discard the device after use, in contrast to a sanitizing wipes and other containers. In this manner, the bracelet sanitation sprayer overcomes the limitations of methods used to disinfect exposed contact surfaces.

### SUMMARY OF THE INVENTION

The following presents a simplified summary in order to provide a basic understanding of some aspects of the disclosed innovation. This summary is not an extensive overview, and it is not intended to identify key or critical elements or to delineate the scope thereof. Its sole purpose is to present some general concepts in a simplified form as a prelude to the more detailed description that is presented later.

The subject matter disclosed and claimed herein, in one embodiment thereof, comprises a sanitation spray bracelet having a hollow body that has a side surface with an opening, a top surface with a spray nozzle and an internal tubing. The opening of the side surface allows the invention to be worn around the wrist of the user. The top surface has a spray nozzle that may be fixedly or removably-attached to the body. In a removable embodiment, the base attaches to a protrusion of the top surface that has a plurality of threads which engage a plurality of corresponding threads located within the base. The top surface of the nozzle has a groove that receives a finger of a user when a user presses down on the nozzle, thereby creating a suction to draw fluid from a reservoir.

The side surface of the nozzle has a continuous opening that allows a sanitizing solution stored within a reservoir contained within the body to be sprayed onto nearby surfaces. To allow the solution to reach the nozzle, a hollow tubing is disposed within the body and removably-attaches to the nozzle. The sanitizing solution may include any cleaning compound, sanitizing compound or disinfection compound such as, but not limited to, a soap, hand sanitizer, or any other disinfecting or sanitizing product, agent, compound or combinations thereof that are in liquid form and can be sprayed in liquid or aerosol form. The nozzle can further be stored within a cap when not in use. The cap may further be retained onto the side surface and/or the base via a tether, so that it does not become lost when not in use. When desired, a user can remove the cap and press down on the nozzle with his or her finger in order to spray solution onto a surface such as, but not limited to, a doorknob, a shopping cart handle, a push bar or any other surface a user may come into contact with during his or her daily life. In this manner, a user can ensure any surface is sanitized before he or she touches the surface.

Accordingly, the bracelet sanitation sprayer of the present invention is particularly advantageous, as it allows a user to easily sanitize every day and other surfaces via a device that can be worn conveniently on the body of the user. As a result, the device does not have to be stored in a pocket, purse or bag. Further, the device does not require a user to discard the bracelet after use, in contrast to a sanitizing wipe. In this manner, the bracelet sanitation sprayer overcomes the limitations of methods used to disinfect surfaces known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one potential embodiment of a bracelet sanitation sprayer of the present invention in accordance with the disclosed architecture;

FIG. 2 illustrates an enhanced perspective view of one potential embodiment of the bracelet sanitation sprayer of the present invention in accordance with the disclosed architecture;

FIG. 3 illustrates an enhanced perspective view of one potential embodiment of the bracelet sanitation sprayer of the present invention in accordance with the disclosed architecture; and

FIG. 4 illustrates a perspective view of one potential embodiment of a bracelet sanitation sprayer of the present invention being worn by a user in accordance with the disclosed architecture.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a device that allows a user to easily sanitize every day and other contact surfaces. There also exists a long-felt need in the art for a convenient sanitation spray that can be easily worn around a human appendage, and allows a user to easily sanitize every day and other contact surfaces and such that the device does not have to be stored in a pocket, purse or bag. Further, there exists a long-felt need in the art for a wrist-worn sanitation sprayer device that does not require a user to discard the device after use, in contrast to a sanitizing wipe or sanitizing bottle.

The present invention, in one exemplary embodiment, is comprised of a wrist-worn sanitation sprayer having a hollow body that has a side surface with an opening, a top surface having a spray nozzle and an internal tubing to draw solution from a reservoir to the spray nozzle. The opening of the side surface allows the device to be worn around the wrist of the user. The top surface has a spray nozzle that may be fixedly or removably-attached to the body. In a removable embodiment, the base attaches to a protrusion on the top surface that has a plurality of threads which engage a plurality of corresponding threads located within the base. The top surface of the nozzle has a groove that receives a finger of a user when a user presses down on the nozzle. The pump action created by depressing the nozzle draws a solution from the reservoir which is then dispensed out of the spray nozzle.

The side surface of the nozzle has an opening that allows a sanitizing solution that is stored within a reservoir within the body to be sprayed onto nearby surfaces. A hollow tubing within the body allows the solution to reach the nozzle. The sanitizing solution may include any cleaning compound, sanitizing compound or disinfection compound such as, but not limited to, a soap, hand sanitizer or any other disinfecting or sanitizing product, agent, compound or other combination that is in liquid form and can be sprayed in liquid or aerosol form. The nozzle can further be stored and protected by a cap when not in use. The cap may further be retained onto the side surface and/or the base via a tether. To use the device, a user can remove the cap and press down on the nozzle with his or her finger in order to spray solution onto a surface such as, but not limited to, a doorknob, a shopping cart handle, push bar or any other surface a user may come into contact within their daily life. In this manner, a user can ensure any surface is sanitized before they touch said surface.

Accordingly, the sanitation spray bracelet device of the present invention is particularly advantageous as it allows a user to easily sanitize every day and other surfaces they come into contact with via a device that can be worn on the body of the user. As a result, the device does not have to be stored in a pocket, purse or bag. Further, the device does not require a user to discard the device after use, in contrast to a sanitizing wipe or bottle of sanitizing solution. In this manner, the wrist-worn sanitation sprayer overcomes the limitations of methods used to disinfect surfaces known in the art.

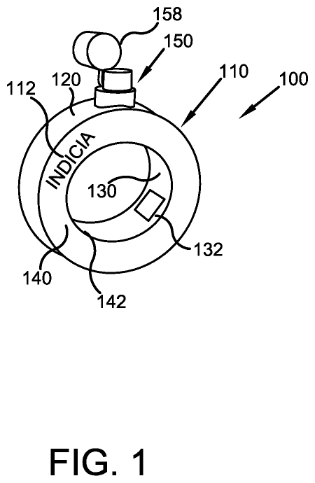

Referring initially to the drawings, FIG. 1 illustrates a front perspective view of one potential embodiment of a wrist-worn sanitation sprayer 100 of the present invention in accordance with the disclosed specification. The invention 100 includes a hollow body 110 that has a side surface 140 with an opening 142, a top surface 120 with a spray nozzle 150 and an internal tubing 160. The body 110 of the device 100 is circular in shape, such that it can fit on or around the wrist 12 of a user. To allow the body 110 to fit around the wrist 12 of a user, the side surface 140 has an opening 142. In differing embodiments, the body 110 and/or opening 142 may be of various sizes to fit around the wrist 12 of differing users, such as adult men and women, teenagers and children. The body 110 may further be transparent, semi-transparent or opaque, and may be of any color known in the art. Further, any surface 120, 130, 140 of the body 110 may have a plurality of indicia 112 such as but not limited to: patterns, logos, emblems, images, symbols, designs, letters, words, characters, animals, advertisements, brands, or combinations thereof. In addition, the bottom surface 130 may further include a padding to increase comfort to the wrist 12 of a user. Furthermore, the device 100 and all components are preferably manufactured from a flexible material, a semi-rigid material, or rigid material such as plastic that may have elastic properties such as, but not limited to, acrylic, polycarbonate, polyethylene, thermoplastic, acrylonitrile butadiene styrene, low density polyethylene, medium density polyethylene, high density polyethylene, polyethylene terephthalate, polyvinyl chloride, polystyrene, polylactic acid, acetal, nylon, fiberglass, recycled plastic, biodegradable plastic, etc.

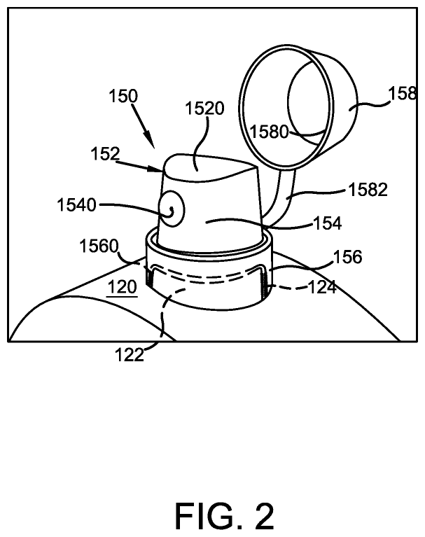

FIG. 2 illustrates an enhanced perspective view of one potential embodiment of a wrist-worn sanitation sprayer 100 of the present invention in accordance with the disclosed specification. The top surface 120 is has a spray nozzle 150 for dispensing solution onto a surface. In differing embodiments, the nozzle 150 may be fixedly-attached to the body 110 or removably-attached to the body 110. In a removable embodiment, the base 156 attaches to a protrusion 122 extending upwardly from the top surface 120. The protrusion 122 has a plurality of threads 124 which engage a plurality of corresponding threads 1560 located within the base 156 of the nozzle 150. The top surface 152 of the nozzle 150 has a groove 1520 that receives a finger 14 of a user. The side surface 154 of the nozzle 150 has a continuous opening 1540 that allows a sanitizing solution 162 that is stored within the body 110 to be sprayed onto nearby surfaces 10.

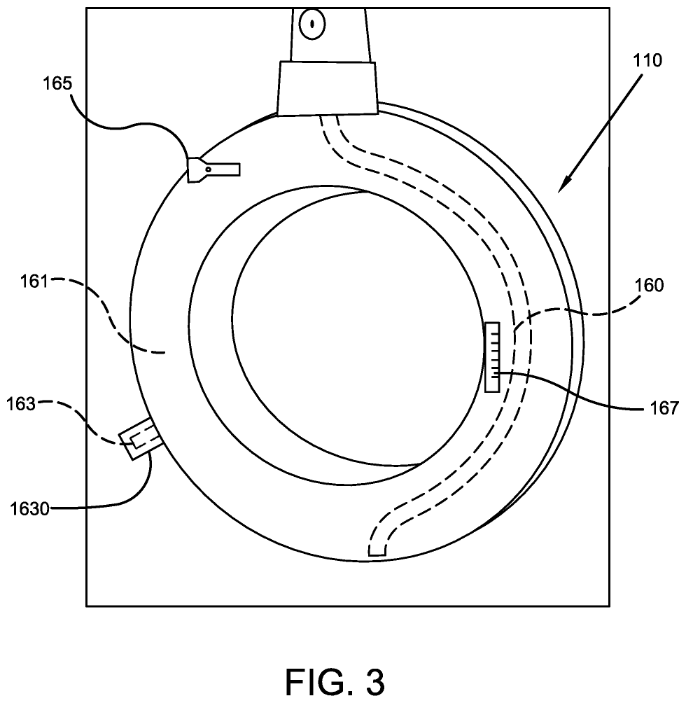

To allow the solution 162 to reach the nozzle 150, a hollow tubing 160 is present within the reservoir 161 of the body 110 that removably attaches to the nozzle 150, as seen in FIG. 3. In differing embodiments, the sanitizing solution 162 may include any cleaning compound, sanitizing compound or disinfection compound such as, but not limited to, a soap, hand sanitizer, or any other disinfecting or sanitizing product, agent, compound or other combinations of solutions that is in liquid form and can be spray in liquid or aerosol form. The nozzle 150 can further be stored within an opening 1580 of a cap 158 when not in use. In one embodiment, the cap 158 may be retained onto the side surface 154 and/or the base 156 via a tether 1582. The body 110 may also include a drainage port or dispensing nipple 163, which can be used to not only drain the reservoir but also to allow the user to pour or dispense the solution onto a larger area as opposed to having to make repeated pumping of the nozzle. The nipple 163 may further have a removable cap 1630 that can be placed over the nipple 163 when not in use. When using a nipple, flexing the nipple will allow the solution to drip. The body 110 may also include a light source 165 so that the user can see in dimly lit areas that he or she is spraying the desired areas. In addition, the light source may be a UV light which can detect certain types of pathogens. The body may further include a gauge 167 so that the user can determine the amount of solution remaining in the reservoir. In other embodiments, the cap may be a screw-on or a snap-on cap 158.

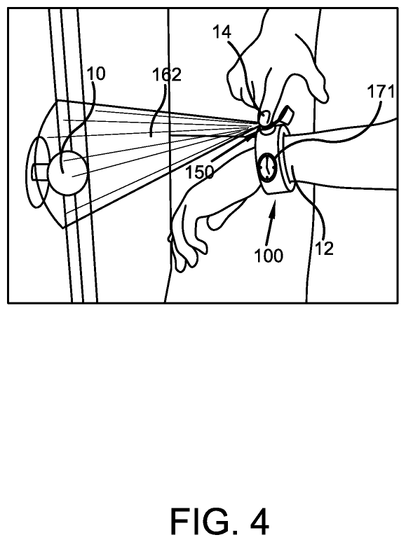

FIG. 4 illustrates a perspective view of one potential embodiment of a sanitation spray bracelet device 100 of the present invention while being worn by a user in accordance with the disclosed specification. As noted, the invention 100 can be worn on the wrist 12 of a user. When desired, a user can remove the cap 158 and press down on the nozzle 150 with his or her finger 14 in order to spray solution 162 onto a surface 10 such as, but not limited to, a doorknob, a shopping cart handle, door push bar or any other surface a user may come into contact within their daily life. In this manner, a user can ensure any surface 10 is sanitized of germs, bacteria, viruses and pathogens before they touch said surface 10. The body 110 may further include a timing mechanism such as a clock, stopwatch or alarm 171, thereby allowing the user to tell time or to be used as an alarm to tell the user when it is safe to touch the surface on to which the solution was sprayed. The alarm or stopwatch can be triggered by depressing the nozzle. The clock may be analog or digital.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “sanitation spray bracelet device” and “device” are interchangeable and refer to the sanitation spray bracelet device 100 of the present invention.

Notwithstanding the forgoing, the sanitation spray bracelet device 100 of the present invention and its various components can be of any suitable size and configuration as is known in the art without affecting the overall concept of the invention, provided that they accomplish the above-stated objectives. One of ordinary skill in the art will appreciate that the size, configuration and material of the sanitation spray bracelet device 100 as shown in the FIGS. are for illustrative purposes only, and that many other sizes and shapes of the sanitation spray bracelet device 100 are well within the scope of the present disclosure. Although the dimensions of the sanitation spray bracelet device 100 are important design parameters for user convenience, the sanitation spray bracelet device 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A personal sprayer comprising:
a body having a reservoir to contain a solution, wherein the body comprises an outer surface with a projection extending upwardly from the outer surface and a nozzle positioned over the projection;
an opening in the body, wherein the opening is sized and configured to accommodate a human wrist, such that the body encircles the human wrist;
a flexible tube extending from the nozzle to the reservoir; and
the nozzle comprising a spray pump configuration to dispense the solution from the reservoir after depressing the nozzle.

2. The personal sprayer as recited in claim 1, wherein the body further comprises a nipple dispenser for dispensing the solution from the reservoir.

3. The personal sprayer as recited in claim 1, wherein the solution is a sanitizing solution.

4. The personal sprayer as recited in claim 3, wherein the sanitizing solution comprises at least one of a cleaning compound, a sanitizing compound a disinfecting compound or a combination thereof.

5. The personal sprayer as recited in claim 1, wherein the body further comprises a timing mechanism.

6. The personal sprayer as recited in claim 5, wherein the timing mechanism comprises at least one of a clock, a stopwatch or an alarm.

7. The personal sprayer as recited in claim 6, wherein the timing mechanism is activated upon a depression of the nozzle.

8. The personal sprayer as recited in claim 1, wherein the body further comprises a light.

9. The personal sprayer as recited in claim 8, wherein the light is a UV light.

10. The personal sprayer as recited in claim 8, wherein the light is used to illuminate a surface for treatment in a dimly illuminated area.

11. The personal sprayer as recited in claim 1, wherein the body further comprises a gauge for determining a level of the solution remaining in the reservoir.

12. A wrist worn sanitizing device comprising:
a body comprising a continuous opening, an interior reservoir and an exterior surface, wherein the continuous opening is sized and configured to be receive a wrist of a wearer;
a sanitizing solution contained in the interior reservoir;
a spray nozzle provided on the exterior surface of the body, wherein the spray nozzle is connected to a flexible tube running from the spray nozzle to a bottom portion of the reservoir, and further wherein the spray nozzle has a pump action to withdraw the sanitizing solution from the reservoir to dispense through the spray nozzle; and
a cap connected to a tether.

13. The wrist worn sanitizing device as recited in claim 12, wherein the body further comprises a nipple valve for allowing the sanitizing solution to be removed from the reservoir.

14. The wrist worn sanitizing device as recited in claim 12, wherein the sanitizing solution comprises a select one of a cleaning compound, a sanitizing compound, a disinfecting compound or a combination thereof.

15. The wrist worn sanitizing device as recited in claim 12, wherein the body is comprised of a transparent material, a semi-transparent material or an opaque material.

16. The wrist worn sanitizing device as recited in claim 12, wherein the body is manufactured from a flexible material, a semi-rigid material, or a rigid material.

17. The wrist worn sanitizing device as recited in claim 12, wherein the body comprises an indicia selected from a group consisting of a pattern, a logo, an emblem, an image, a symbol, a design, a letter, a word, a character, an animal, an advertisement, a brand or a combination thereof.

18. The wrist worn sanitizing device as recited in claim 12, wherein the body further comprises a timing mechanism and a light.

19. The wrist worn sanitizing device as recited in claim 18, wherein the timing mechanism is activated by depressing the spray nozzle.

20. A bracelet sanitation sprayer comprising:
a bracelet comprising a continuous opening therein, an outer surface and an inner reservoir for holding a sanitizing solution, wherein the continuous opening is sized and configured to accommodate a wrist of a wearer;
a spray nozzle disposed on the outer surface of the bracelet, wherein the spray nozzle is connected to a flexible tubing extending from the nozzle to a bottom of the reservoir, wherein the spray nozzle is a pump action spray nozzle to draw the sanitizing solution from the reservoir to the spray nozzle;
a cap tethered to the bracelet;
a timing mechanism;
a gauge; and
a light.
